(19) United States
(12) Patent Application Publication
(10) Pub. No.: US 2022/0168139 A1
(43) Pub. Date: Jun. 2, 2022

(54) DEVICE FOR THE TREATMENT OF A MYOCARDIAL INFARCTION

(71) Applicant: UBITEMP, WATERLOO (BE)

(72) Inventors: Eric JEAN, BIEVILLE-BEUVILLE (FR); Fabrice MISSAIRE, LILLOIS (BE); Sylvain THUAUDET, LE FRESNE-CAMILLY (FR)

(21) Appl. No.: 17/436,904

(22) PCT Filed: Mar. 5, 2020
(86) PCT No.: PCT/EP2020/055919

§ 371 Date: Sep. 7, 2021

(30) Foreign Application Priority Data
Mar. 6, 2019 (FR) FR1902299

(51) Int. Cl. A61F 7/12; A61M 25/10; A61F 7/00

(52) U.S. Cl. CPC A61F 7/123; A61F 2007/0056; A61F 7/007; A61F 7/0085; A61M 2205/3334; A61M 2205/3344; A61M 2205/3368; A61M 2205/3653; A61M 2205/3606; A61M 2205/366; A61F 2007/0086; A61F 2007/0071; A61F 2007/0091; A61F 2007/126; A61M 25/104

(57) ABSTRACT

The invention relates to a device (1) for treating a coronary lesion (2), the device comprising a coronary angioplasty catheter (3) presenting a first lumen (3c) for passing a guide wire (5) for guiding the catheter (3), and at least one balloon (3b) situated at said distal portion and connected to a second lumen for controlling the balloon (3d).

The device further comprises a heat exchanger (6), a pump (7), and at least one first coupling (8a) for coupling the device with a liquid reservoir (R) external to the device (1), the pump (7) being in fluid flow connection with the first coupling (8a), with the heat exchanger (6), and with the first lumen (3c) so as to force a flow of the liquid from the first coupling (8a) to the distal end of the first lumen (3c), after passing through the heat exchanger (R).

The present invention relates to the field of devices usable for treating coronary lesions.

### BACKGROUND OF THE INVENTION

Coronary arteries distributed around the myocardium supply the myocardium with oxygenated blood.

A lesion in a coronary artery can lead to a lack of blood supply to at least a portion of the myocardium, and sometimes to a risk of myocardial infarction.

Typically, a coronary artery lesion is an arteriosclerotic plaque (hereinafter “plaque”) obstructing the artery, at least in part.

The coronary lesion can be treated by an angioplasty seeking to re-establish a satisfactory passage for blood in the artery concerned.

While blood flow is being re-established, the myocardium receives blood loaded with toxins.

The harmful effects of the toxins can be attenuated by slowing down cellular activity by cooling the blood and/or the myocardium.

### OBJECT OF THE INVENTION

An object of the present invention is to provide a device for treating a coronary lesion that enables myocardial lesions to be limited.

### SUMMARY OF THE INVENTION

To this end, in a first aspect of the invention, there is provided a device for treating a coronary lesion, the device comprising a coronary angioplasty catheter presenting a distal portion adapted to penetrate into a coronary artery, the coronary angioplasty catheter presenting a first lumen open at proximal and distal ends of the first lumen to enable a guide wire to pass through the coronary angioplasty catheter and guide it in a coronary artery, the coronary angioplasty catheter also including at least one balloon situated at said distal portion of the catheter, the balloon being connected to a second lumen for controlling the balloon so that the balloon adopts an inflated configuration or a deflated configuration selectively as a function of fluid pressure inside said second lumen.

This device for treating a myocardial infarction is essentially characterized in that it further comprises a heat exchanger, a pump, and at least one first coupling for coupling the device with a liquid reservoir external to the device, the pump being in fluid flow connection with the first coupling, with the heat exchanger, and with the first lumen so as to force a flow of the liquid from the first coupling to the distal end of the first lumen, after passing through the heat exchanger.

A guide wire is previously installed in the artery for treatment and then the angioplasty catheter is guided by sliding along the guide wire, which passes via the first lumen.

The catheter is thus guided so as to position its balloon in register with the lesion for treatment.

The guide wire can be introduced into the coronary artery by causing it to slide in a lumen of a guide catheter that has previously been installed and that leads into the artery presenting the lesion for treatment.

Once the angioplasty catheter is in position in the coronary artery, the balloon can be inflated in order to perform angioplasty and the plaque is then compressed against the wall of the artery.

The angioplasty balloon is inflated or deflated by varying the pressure in the second lumen of the catheter.

By means of the invention, the first lumen, which is normally used solely for passing the guide wire, is now connected to the pump and to the heat exchanger so as to be capable of injecting a volume of liquid into the artery the purpose of varying the temperature of the myocardium and producing a therapeutic effect.

The device of the invention makes it possible:
to infuse a volume of liquid into the artery, downstream from the lesion, which volume of liquid serves to vary the temperature of the myocardium; and
to perform this infusion both at the same time as performing the angioplasty and also after the angioplasty has been performed.

Thus, the temperature of the myocardium can begin being varied as soon as possible, thereby enabling an increased volume of the myocardium to be subjected to the temperature variation.

The therapeutic effect associated with varying the temperature of the myocardium in this way is thus improved and extends over a larger volume of the myocardium.

The cells of the myocardium that are cooled in this way are less sensitive to the toxins that they receive at the time blood begins to flow again, thus serving to limit lesions of the myocardium.

Preferably, he cells of the myocardium are subjected to a cooling temperature variation for the purpose of limiting the harmful effects of toxins contained in the blood at the time the blood begins to flow again.

By cooling the myocardium while the balloon is inflated (during angioplasty), the myocardium is cooled better and over a larger volume, and it is better protected against the effects of toxins.

A significant therapeutic effect is observed when, before blood begins to flow again and for a duration of several minutes after blood has begun to flow again, the temperature of the myocardium is kept below 36° C., preferably in the range 31° C. to 33° C., more preferably at 32° C.

Preferably, the distal portion of the catheter has a length of at least 1 centimeter (cm), and the catheter together with its balloon in the deflated configuration has a maximum outside diameter as measured in its distal portion that is less than or equal to 4 millimeters (mm).

### BRIEF DESCRIPTION OF THE DRAWINGS

Other characteristics and advantages of the invention appear clearly from the following description that is given by way of nonlimiting indication and with reference to the accompanying drawings, in which:

FIG. 1 is a diagrammatic view of the device 1 of the invention together with a console 100 of the invention prior to being used for treating a lesion/plaque in a coronary artery;

FIG. 2 is a diagrammatic view of the device 1 of the invention together with the console 100 of the invention while they are being used to treat a lesion/plaque in a coronary artery 4 of a myocardium 2;

FIG. 3a is a diagrammatic view of a zone A of one of the coronary arteries 4 visible in FIG. 2, shown prior to inserting an angioplasty catheter into the artery 4, a lesion or plaque P is obstructing the artery 4 in part and is opposing the flow of blood towards the portion of the artery that is downstream from the lesion P;

FIG. 3b is a diagrammatic view of the coronary artery 4 of FIG. 3a after passing a guide wire 5, the guide wire 5 extending from a zone outside the patient to a portion of the damaged coronary artery situated downstream from the lesion P, with the wire 5 passing through the lesion P;

FIG. 3c is a diagrammatic view of the coronary artery 4 of FIG. 3a after a coronary angioplasty catheter 3 has been slid around the guide wire 5, the guide wire 5 passing through the first lumen 3c of the catheter, the distal portion 3a of the catheter 3 that includes an inflatable balloon 3b passes through the lesion P, the balloon being deflated and in register with the plaque P for treatment (the balloon is deflated while the catheter is being put into place in the artery and being passed through the lesion);

FIG. 3d is diagrammatic view of the coronary artery 4 of FIG. 3a while angioplasty is being performed by inflating the balloon 3b and while a cooling liquid (physiological serum) is being infused into the portion of the artery downstream from the lesion, this liquid flowing at 20° C. via the first lumen 3c with the temperature of the myocardium downstream from the lesion being lowered to a temperature in the range 32° C. to 33° C.;

FIG. 3e is a diagrammatic view of the coronary artery 4 of FIG. 3a after the angioplasty has been performed, with the balloon 3b being deflated and with blood flowing between the deflated balloon and the plaque P, the cooling liquid (physiological serum) is still being infused towards the portion downstream from the lesion via the first lumen 3c, but its temperature now lies in the range 4° C. to 5° C. in order to keep the temperature downstream from the lesion in the range 32° C. to 33° C. (this limits the temperature rise associated with blood at a temperature in the range 36° C. to 37° C. flowing again);

FIG. 3e is a diagrammatic view of the coronary artery 4 of FIG. 3a after successively performing the angioplasty while cooling the myocardium, guiding a vascular endoprosthesis 40 (also known as a “stent”) into register with the lesion P, expanding the endoprosthesis 40 by inflating the balloon 3b of the catheter 3, deflating the balloon 3b, and removing the catheter 3 so as to leave the endoprosthesis 40 in the artery, with blood flow then being re-established in the artery and the myocardium being back at its temperature of 37° C.; and

FIG. 4 shows the temperature setpoints V1 and V2 for the liquid respectively during a first period P1 in which the balloon is inflated and prevents blood from flowing towards the portion of the artery downstream from the lesion and during a second period P2 during which the balloon is deflated and blood flows towards the downstream portion of the artery, and it shows how the setpoint varies over time during a period P1′ between the periods P1 and P2, with the setpoint varying to go progressively from the setpoint V1 to the setpoint V2, which is lower than the setpoint V1.

### DETAILED DESCRIPTION OF THE INVENTION

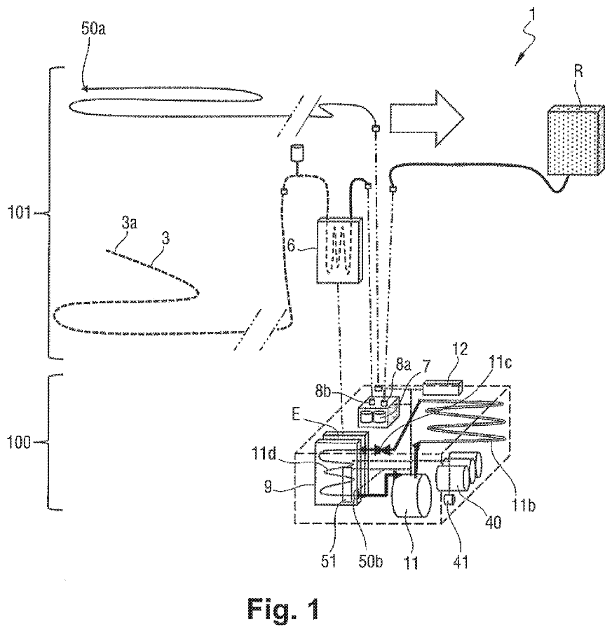

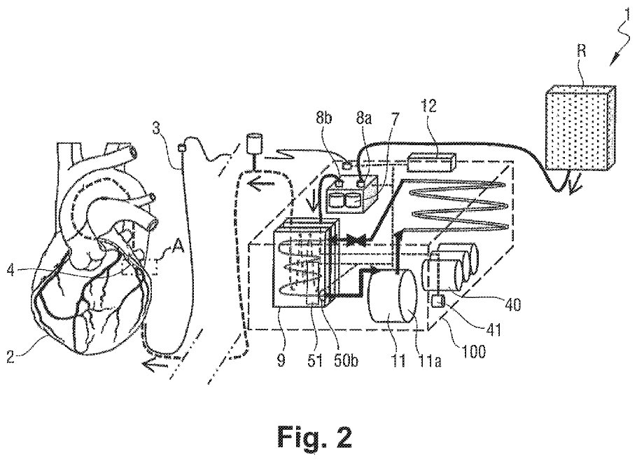

As mentioned above, and as shown in FIGS. 1 and 2, the invention relates to a device 1 for treating a coronary lesion, specifically a plaque P, which might give rise to an infarction of the myocardium 2.

The device 1 has a coronary angioplasty catheter 3 presenting a distal portion 3a adapted to penetrate into a coronary artery 4.

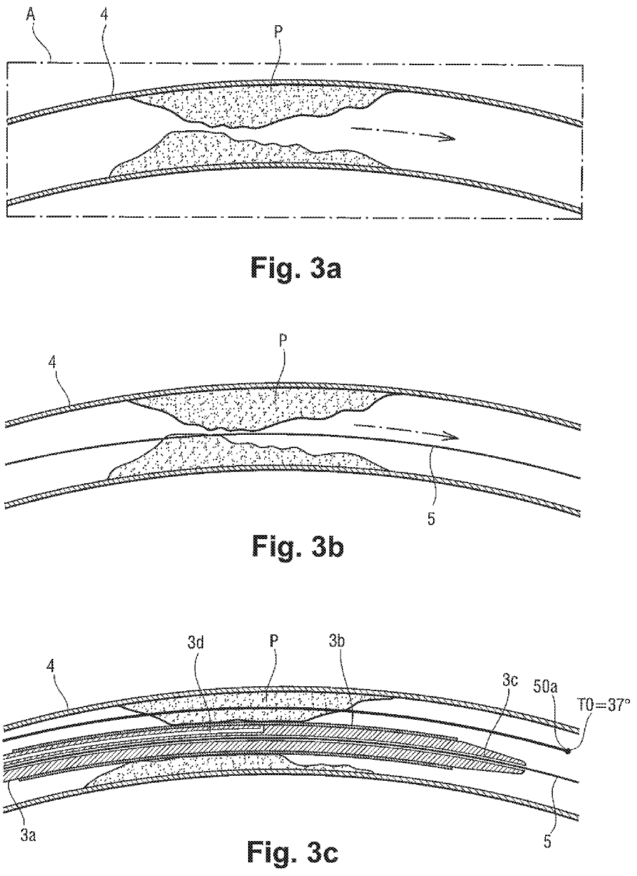

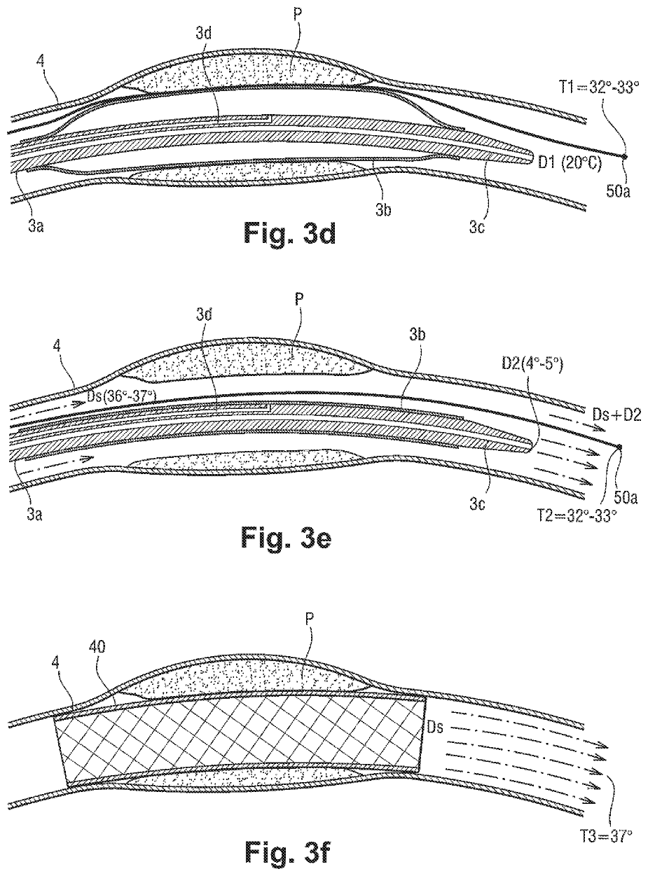

The distal portion presents a tapering tip to facilitate inserting it into the artery 4 and passing it through the lesion P for treatment (see FIG. 3c) while nevertheless avoiding moving the lesion towards a downstream zone of the artery.

In this example, the tip of the catheter is dome-shaped.

The coronary angioplasty catheter 3 presents a first lumen 3c that is open both at its proximal end and at its distal end in order to allow a guide wire 5 to pass through for guiding the coronary angioplasty catheter 3 within the coronary artery 4. This first lumen 3c is preferably a central lumen of the catheter.

The coronary angioplasty catheter 3 also has at least one balloon 3b situated on said distal portion 3a of the catheter 3.

The balloon 3b is connected to a second lumen 3d for controlling the balloon 3b, which lumen opens out at one end into the balloon and at its other end to the outside of the catheter in a proximal portion of the catheter.

Admission means (not shown) for admitting liquid into the balloon are connected to the proximal end of the second lumen 3d in order to be able to admit a fluid/liquid under pressure for inflating the balloon.

The balloon is preferably elastic so that it deflates when the pressure in the second lumen 3d is reduced.

Thus, as a function of the pressure of fluid inside said second lumen 3d, the balloon 3b selectively adopts an inflated configuration or a deflated configuration.

The device 1 also has a heat exchanger 6, a pump 7 (in this example a gear pump), and at least one first coupling 8a for coupling the device to a liquid reservoir R external to the device 1. The liquid is preferably a physiological serum.

The external reservoir R may contain a volume of liquid that is sufficient for supplying liquid to the device 1 over at least 20 minutes with the liquid flowing at a rate of at least 20 milliliters per minute (mL/min). Thus, a volume of at least 400 mL of heat exchange liquid may be admitted into the myocardium over 20 minutes. The liquid reservoir preferably contains 0.5 liters (L) to 2 L of liquid (physiological serum).

As can be seen in FIG. 2, the pump 7 is in fluid flow connection with the first coupling 8a, with the heat exchanger 6, and with the first lumen 3c so as to force the liquid to flow from the first coupling 8a (which is connected to the reservoir R) towards the distal end of the first lumen 3c after passing through the heat exchanger 6.

By means of the invention, during angioplasty, while the balloon 3b is in its inflated configuration so as to obstruct the artery at its lesion P, liquid is infused into the coronary artery 4 downstream from the lesion P in order to cause the temperature of the myocardium downstream from the lesion to vary.

Varying the temperature of the myocardium serves to obtain therapeutic effects that are particularly beneficial for the patient.

In particular, as mentioned above, lowering the temperature of the myocardium to a temperature in the range 32° C. to 33° C. serves to reduce the harmful effects of toxins on the myocardium when blood begins to flow again.

The pump serves to pump the liquid from the liquid reservoir R external to the device 1 so as to force it to flow through the heat exchanger 6 on its way to the coronary artery 4 into which it is diffused.

The temperature of the liquid is adapted continuously by using the heat exchanger 6, without any need to disconnect lines and without any need to keep the reservoir R in a temperature-regulated location.

By means of the invention, even if the liquid in the reservoir R is stored at room temperature, in the range 18° C. to 22° C., its temperature is adapted directly by the device 1, and it can be infused rapidly into the patient's coronary artery.

The presence of a liquid line including a pump 7 and a heat exchanger 9 coupled together in series makes it possible to control the flow rate and/or the temperature of the fluid being infused into the artery 4 both continuously and accurately. This improves accuracy and safety for the patient.

Preferably, the distal portion 3a of the catheter has a length of at least 1 cm, and the catheter together with its balloon 3b in the deflated configuration has a maximum outside diameter as measured in its distal portion that is less than or equal to 4 mm.

This maximum outside diameter is 4 mm (see FIG. 3c), and preferably it lies in the range 1.5 mm to 0.5 mm, which facilitates inserting the distal portion of the catheter through the lesion P.

This outside diameter in the range 1.5 mm to 0.5 mm makes it possible to treat lesions in artery portions having a diameter less than 2 mm.

The distal portion 3a of the catheter 3 is at least 1 cm long, and preferably it is at least 5 cm long, more preferably at least 25 cm long.

Since the maximum length of a coronary artery is generally 25 cm, a distal portion 3a having a length of at least 25 cm is adapted to penetrate along the entire length of a coronary artery in order to treat lesions therein.

Ideally, the total length of the catheter is greater than 1 meter (m), and preferably greater than or equal to 1.8 m.

The distal end of the catheter 3 is preferably pointed so as to facilitate inserting it into the artery and causing it to penetrate through the lesion.

The first lumen 3c for passing the guide wire 5 preferably has an inside diameter that is less than or equal to 1 mm, more preferably the inside diameter lies in the range 0.5 mm to 0.1 mm, still more preferably in the range 0.5 mm to 0.3 mm, ideally it is 0.36 mm.

The balloon 3b extending at the periphery of the catheter 3 over its distal portion 3a ideally presents a balloon length lying in the range 8 mm to 40 mm.

Since the inside diameter of the first lumen 3c is very small, the pump needs to present considerable delivery pressure in order to deliver liquid at a flow rate that is sufficient to cool the myocardium.

To do this, the pump is preferably a gear pump adapted to increase the pressure of the liquid that it drives by at least 10 bars, preferably at least 20 bars, more preferably at least 30 bars.

This high pressure makes it possible to achieve high flow rates with a first lumen of very small inside diameter, generally less than 0.5 mm.

Thus, the pump 7 presents a liquid delivery pressure to the first lumen of the catheter of at least 1000 kilopascals (kPa) (i.e. about 10 bars), preferably at least 2000 kPa (i.e. about 20 bars).

This delivery pressure and the flow rate of the pump are adapted to give rise to a liquid flow rate D1, D2 via the first lumen 3c of at least 10 mL/min (ideally of at least 15 mL/min, which is appropriate for injecting into a coronary artery section with a diameter of less than 2.5 mm, more preferably of at least 20 mL/min, which is adapted to injecting into a coronary artery section with a diameter lying in the range 4 mm to 2.5 mm).

This flow rate D1, D2 of at least 20 mL/min, preferably of at least 30 mL/min, is considered to be sufficient for cooling a sufficient volume of the myocardium to 32° C., including when blood at 37° C. begins to flow again.

Preferably, the device 1 also has at least one temperature variation element 9 adapted to be associated with said at least one heat exchanger 6 in order to vary the temperature of the liquid passing through the heat exchanger 6.

For a liquid flowing through the heat exchanger with a liquid flow rate in the heat exchanger lying in the range 10 mL/min to 40 mL/min, and preferably in the range 20 mL/min to 30 mL/min, this at least one temperature variation element is adapted to cause the temperature of that liquid to vary with a gradient of up to 10° C., preferably up to 20° C., more preferably up to 30° C., still more preferably up to 40° C.

Thus, by using both the heat exchanger 6 and the temperature variation element 9, the capacity for heat exchange is sufficient to enable the temperature of the liquid flowing through the heat exchanger 6 to be lowered by a temperature gradient of up to 40° C., with a liquid of the physiological serum type flowing at a rate of up to 40 mL/min.

The device of the invention has the capacity for lowering the temperature of the liquid by at most 40° C. while the liquid is flowing at a rate of at most 40 mL/min.

This capacity is extreme, and enables the device to be used even in situations where the liquid reservoir R is installed in premises that are not air-conditioned, at a temperature that might be as much as 40° C.

In most situations, the device has cooling capacity that is greater than requirements, thus enabling it to operate at a lower rate, which is favorable to supplying the myocardium safely with cooled liquid.

With reference to FIGS. 1 and 2, the device 1 also has a control unit 12 and at least one temperature probe 50a, 50b arranged to communicate at least one measured temperature value to the control unit 12, which measured temperature value is representative of the temperature of the flowing liquid.

The temperature probe 50b may be arranged to measure a temperature value upstream from the catheter 3. This serves to have a first temperature measurement that is relatively independent of the temperature of the surroundings of the catheter, and consequently relatively independent of the patient's body temperature.

In this embodiment, the temperature value is preferably measured at the outlet from the heat exchanger 6. Ideally, the temperature probe is mechanically connected to said at least one temperature variation element 9 so as to enable the heat exchanger to be removed while leaving the probe permanently with the temperature variation element 9. Even when the probe is mechanically connected to the temperature variation element 9, its position is chosen so as to measure a temperature that is representative of the outlet temperature from the heat exchanger 6 upstream from the catheter.

The heat exchanger 6 may possibly include a pressure probe arranged to transmit a value to the control unit 12 for the pressure of the liquid in the heat exchanger.

Likewise, the pump 7 may possibly include a pressure probe arranged to transmit a value to the control unit 12 for the pressure at which liquid is being delivered to the catheter.

The temperature probe may also be arranged so that said measured temperature value is measured between the heat exchanger and the distal end of the first lumen.

In this embodiment, the probe may be incorporated in the catheter, which facilitates installing and/or positioning it relative to the catheter.

More particularly, the temperature probe may be located at the distal end of the catheter. The temperature probe may optionally be attached to a pressure probe that is likewise arranged at the distal end of the catheter.

It is also possible for the temperature probe 50a to be located at the end of a wire arranged to run along and outside the catheter 3 so as to enable the temperature probe 50a to be positioned downstream from the distal end of the catheter (3) (i.e. downstream from the lesion P through which the distal portion of the catheter passes).

With the probe 50a installed in this way, as shown in FIGS. 1, 2, 3c, 3d, and 3e, temperature is measured downstream from the lesion, i.e. where it is desired to guarantee that the temperature of the myocardium is regulated.

This serves to improve the accuracy of regulation, both at the time when the flow of blood is stopped and at the time when it begins again.

The wire with the temperature probe 50a may be a guide wire, possibly fitted with at least one pressure probe at its distal end.

Each of these probes communicates with the control unit 12 in order to transmit measured temperature and pressure values. The measured temperature value may be a value measured using a plurality of temperature probes 50a, 50b).

There may be a plurality of pressure probes for measuring a plurality of pressures of the liquid flowing through different locations in the device, these pressure measurements being used to regulate the flow rate of the pump and to identify possible head losses and generate warnings in the event of the flow of liquid being obstructed or in the event of a leak.

Likewise, it is possible to have a plurality of pressure probes for measuring a plurality of pressures flowing at various locations around the catheter. These pressure measurements may be used for monitoring blood flow rate, the effectiveness of the closure obtained with the balloon, and possible blockages of the artery or risks of excessive arterial pressure.

These pressure measurements may be used to generate warnings.

The control unit 12 is functionally connected to the pump 7 and to the temperature variation element 9 associated with the heat exchanger 6 in order to control variation of at least one parameter of the flowing liquid in order to achieve a predetermined setpoint for transfer of heat to the catheter 3 via the flowing liquid, which parameter is selected from the temperature of the flowing liquid and the flow rate of the flowing liquid.

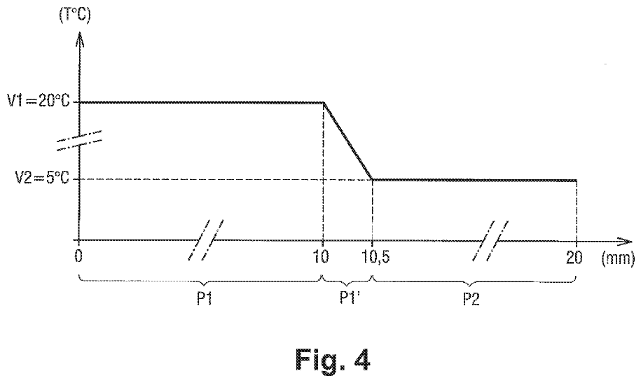

As can be understood from FIG. 4, the control unit 12 is preferably arranged to control said at least one temperature variation element 9 associated with said heat exchanger 6 so that the liquid flowing through the first lumen 3c of the catheter presents a temperature over a first period P1 of several minutes that is equal to a first predetermined temperature setpoint value V1 to within plus or minus 2° C., and a temperature over a second period P2 of several minutes after the first period P1 that is equal to a second predetermined temperature setpoint value V2 to within plus or minus 2° C., the second temperature setpoint value V2 being lower than the first temperature setpoint V1 by at least 10° C., preferably at least 15° C.

The first setpoint value V1 serves to define the temperature of the liquid while the balloon is closing the artery and no blood is flowing to the artery portion downstream from the lesion.

The second setpoint value V2 serves to define the temperature of the liquid while the balloon is in its deflated configuration and while blood is flowing to the artery portion downstream from the lesion.

Using these two setpoints serves to guarantee cooling quality throughout angioplasty and after angioplasty.

These setpoints V1 and V2 are preferably constant while the flow rate of the liquid flowing through the lumen 3c is likewise constant, e.g. in the range of 15 mL/min to 30 mL/min, preferably 20 mL/min.

Typically, the first temperature setpoint value V1 lies in the range 15° C. to 25° C. and the second temperature setpoint value V2 lies in the range 2° C. to 10° C.

As can be seen in FIG. 4, the first setpoint may be applied over the period P1 for 10 minutes, and the second setpoint V2 may be applied over a second period P2 later than the first period P1. This second period P2 is generally 10 minutes long.

Preferably, the control unit 12 and the temperature variation element 9 are arranged so that the liquid flowing through the heat exchanger 6 can reach the second temperature setpoint value V2 less than one minute after reaching the first temperature setpoint C1.

Thus, the control unit 12, the pump 7, and the temperature variation element 9 are designed so that, within the limit of the liquid flow rate that can be delivered by the pump 7, the rate at which the temperature of the liquid changes from its first setpoint value V1 to its second setpoint value V2 is at least 5° C. per minute (to go from 15° C. to 10° C. in less than one minute) and is at most 23° C. per minute (to go from 25° C. to 2° C. in less than one minute).

Preferably, this rate at which the temperature of the liquid changes from its first setpoint value V1 to its second setpoint value V2 lies in the range 7° C. to 15° C. per minute, and is preferably 10° C. per minute.

In order to accommodate thermal inertia and/or any limits of the device, and in order to preserve both the device and the patient, an intermediate period P1′ may be provided between the periods P1 and P2 so as to make it possible to pass progressively from the setpoint V1 to the setpoint V2.

In this example, the intermediate period P1′ starts at time 10 minutes (at the end of P1) and ends at time 10.5 minutes (at the beginning of P2).

The device of the invention may have other probes that communicate with the control unit 12, such as a bubble sensor that is incorporated between the distal end of the catheter and a debubbler that is installed downstream from the heat exchanger 6 and the pump 7.

In the event of bubbles being detected, the control unit 12 generates a bubbles-present warning, and it may optionally cause the pump to be stopped.

The device 1 may also include an atmospheric pressure sensor, with control of the pump 7 being designed so that the pressure of the liquid in the catheter or in the heat exchanger does not exceed a maximum authorized pressure threshold relative to atmospheric pressure as measured by the atmospheric pressure sensor.

The device of the invention may also include a human/machine interface having at least one screen and/or printer and/or audible or vibratory alarms so that the control unit can transmit information to a user. This information may comprise values measured by the probes or setpoint values or duration values or states of portions of the device.

The human/machine interface may also have a keyboard and/or buttons and/or pointing devices and/or voice control means for communicating instructions to the control unit 12. An instruction may be a manual command for varying a flow rate setpoint and/or a temperature setpoint T° C. for the flowing liquid.

As can be understood from FIGS. 1 and 2, said at least one temperature variation element 9 may include an electrical heater resistance 51 suitable for raising the temperature of the liquid quickly and for improving the accuracy with which the temperature of the liquid is regulated.

One use for such a resistance may be to increase the temperature of the liquid until it reaches the setpoint V1 or V2.

Thus, if the liquid is stored in a reservoir at 2° C., the flowing liquid can be heated up to V1=20° C. over the period P1, and up to V2=5° C. over the period P2.

As can be understood from FIGS. 1 and 2, said at least one temperature variation element 9 may also include at least one refrigeration unit 11 comprising a compressor 11a, a condenser lib, an expander 11c, and an evaporator 11d that are connected together to define a closed circuit in which a refrigerant fluid flows, said heat exchanger 6 being arranged to be thermally coupled with said evaporator 11d in order to cool the liquid flowing through said heat exchanger.

The various elements of the device (human/machine interface, heater resistance 51, compressor 11a, control unit 12, valves, sensors, probes) may be electrically powered via one or more batteries 40 (so that the device can operate independently of mains) and/or via an external power outlet 41.

The device may incorporate a battery charger circuit using electricity coming from the outlet 41.

The evaporator 11d is preferably incorporated in an enclosure E for receiving the heat exchanger 6.

The temperature variation element 9 may form an enclosure E in which the heat exchanger 6 can be removably positioned (via an opening in the enclosure E).

Thus, the heat exchanger 6 may be in the form of a heat exchange cassette that is removable from the enclosure.

A cover that is movable relative to the enclosure E may close it, at least when the heat exchanger 6 is inside the enclosure.

This cover limits heat transfer between the inside and the outside of the enclosure E. This improves the efficiency of the device and of the console. For this purpose, the cover may include a layer of insulation and gaskets for sealing the opening of the enclosure.

Preferably, the temperature variation element 9 includes at least two heat exchange faces positioned inside the enclosure so that when the heat exchanger 6 is positioned inside the enclosure E, it is located between these two heat exchange faces.

Ideally, the temperature variation element 9 is surrounded by thermal insulation to limit heat losses and to improve heat exchange between the temperature variation element 9 and the heat exchanger 6.

It should be observed that the heat exchanger 6 and the temperature variation element 9 as described above can be used in any device that delivers a liquid to a catheter 3 for the purpose of giving rise to a change of temperature in at least a portion of a patient.

The couplings 8a, 8b and any couplings that are present between the debubbler and a catheter may be of various types, however for standardization purposes, they are preferably of the Luer lock type (applying a Luer connection standard including a threaded ring for making the connection safe).

The control unit 12 may be connected to an actuator for managing pressure in the second lumen 3d for controlling deflation of the balloon, the control unit being arranged to cause the temperature variation element 9 to reduce its temperature before the control unit 12 sends a balloon-deflation command to the actuator for managing pressure in the second lumen 3d.

Thus, the control unit anticipates the increase in temperature associated with blood flowing again by causing the temperature of the temperature variation element 9 to lower before blood begins to flow again.

With the thermal inertia for cooling the temperature variation element 9 being known, the control unit 12 can generate the balloon-deflation command:
after a predetermined duration has elapsed since the command for reducing the temperature of the temperature variation element 9; and/or
after a probe for measuring the temperature of the temperature variation element has detected a predetermined criterion representative of an expected reduction in the temperature of the temperature variation element 9.

By means of this characteristic, any risk of having a large rise in temperature downstream from the lesion P when blood begins to flow again is limited.

In an additional configuration, the control unit 12 may be connected to a pressure sensor in the second lumen 3d to detect inflation or deflation of the balloon and, as a function of the pressure in the second lumen, to control variation of the temperature of the liquid flowing through the first lumen 3c by automatically controlling the temperature variation element 9 and/or the pump 7 so as to vary the flow rate of liquid.

Thus, it is possible to have an automatic temperature-lowering command followed by an automatic balloon-deflation command (making it possible to take account of the cooling time or the thermal inertia of the heat exchanger and to anticipate the command for cooling it prior to causing blood to flow again, which causes heat to be delivered with the new flow of blood). This limits any risk of having an unwanted temperature rise of the myocardium over the period when blood begins to flow again.

As can be understood from FIGS. 1 and 2, the device of the invention is made up of a console 100 and one or more consumables 101 comprising at least the catheter 3, possibly the heat exchanger 6, the debubbler, the reservoir R, and the wire, if any, connecting the external probe 50a to the console 100.

Thus, in a second aspect, the invention may provide a console 100 comprising:
a temperature variation element 9 arranged to receive a removable heat exchanger 6 of the console 100, the temperature variation element 9 being arranged to obtain heat exchange between the temperature variation element 9 and the heat exchanger 6 received in the temperature variation element 9;
at least one fluid flow driver device, specifically a pump 7 or optionally a syringe pusher or a peristaltic pump;
at least one first coupling 8a for coupling the console 100 with a liquid reservoir R external to the console 100; and
at least one second coupling 8b for coupling the fluid flow driver device 7 (the pump 7) with a heat exchanger 6 external to the console, the fluid flow driver device 7 (the pump 7) of the console 100 being in fluid flow connection with the first coupling 8a and with the second coupling 8b so as to be capable of forcing liquid to flow from the first coupling 8a to the second coupling 8b.

When the fluid flow driver device is a pump 7, then the fluid flows from the first coupling 8a to the second coupling 8b by passing via the pump.

The heat exchanger 6 is not part of the console, and it is a heat exchanger as described above with reference to the device 1 of the invention.

In particular, the heat exchanger 6 is adapted to be coupled to the external catheter 3, e.g. to a coronary angioplasty catheter 3. The external catheter 3 enables the liquid passing via the heat exchanger 6 to flow to the patient.

The temperature variation element 9 may be identical to any of the embodiments described above.

The console includes a support structure on which the following are mechanically connected: the pump 7, a refrigeration unit 11, and the temperature variation element 9 together with a control unit 12 for controlling the console and identical to the unit described above. The console preferably includes batteries and the outlet 41 described above, the batteries and the outlet also being supported by the support structure of the console.

The pump, the refrigeration unit 11, the temperature variation element 9, the control unit 12, the batteries 40, and the outlet 41 all forming parts of the console, and their respective functions and interactions are as described above when describing the device 1.

The console 100 also presents an external shell in which the pump, the element 9, and the refrigeration unit 11 are placed, the ports 8a, 8b being accessible from outside the shell.

There follows a description of the method of using the device for treating a coronary lesion.

This method comprises the following succession of steps:
infusing opaque substance into the myocardium in order to obtain medical imagery and discover the position of the lesion for treatment, the type of the lesion for treatment, and the shapes and/or dimensions of the coronary artery upstream and downstream from the lesion, the medical imagery enabling the practitioner to direct and position equipment in the arterial network;
optionally putting a guide catheter into place, the guide catheter then passing via the aorta at least as far as the coronary artery where the lesion is located;
optionally putting a temperature measurement wire 50a and/or a pressure measurement wire into place while using the guide catheter that has optionally been put into place (the measurement wire being of the guide wire type). The measurement wire includes at least one temperature measurement probe for measuring temperature downstream from the lesion for treatment. The guide catheter is very useful for sliding the measurement wire up to the lesion, with the measurement wire passing through the lesion so that said at least one temperature measurement probe is downstream from the lesion;
optionally putting a wire 5 into place for guiding a coronary angioplasty catheter 3;
putting the coronary angioplasty catheter 3 into place by threading it onto the guide wire and/or by engaging it inside the guide catheter, the angioplasty catheter being arranged to pass through the lesion P in such a manner that the balloon 3b of the coronary angioplasty catheter is in register with the lesion for treatment;
inflating the balloon to perform angioplasty and compress the plaque P that has given rise to the lesion;
removing the guide wire from the angioplasty catheter, this removal preferably taking place while the balloon is inflated (thereby avoiding movement of the angioplasty catheter while the guide wire is being removed. Nevertheless the guide wire could equally well be removed before the balloon is inflated);
causing the liquid coming from the liquid reservoir to flow to the distal end of the catheter by passing via the pump 7 and the heat exchanger 6 (to control its temperature as a function of the temperature setpoint to be reached at the myocardium), this first infusion taking place via the first lumen 3c, preferably with liquid of the physiological serum type, at a flow rate lying in the range 15 mL/min to 30 mL/min, preferably 20 mL/min, the liquid preferably lying at a temperature in the range 20° C. to 22° C., with the duration P1 of this first infusion, while the balloon is inflated in the artery, preferably lying in the range 5 minutes to 10 minutes (thereby obtaining cooling of the myocardium before blood begins to flow again); and
inflating the balloon and performing a second infusion of said liquid into the artery via the same circuit (pump, heat exchanger, and first lumen, the second infusion being performed with a liquid at a temperature in the range 2° C. to 7° C., with the flow rate of the liquid being in the range 15 mL/min to 30 mL/min, the second infusion taking place at the same time as blood flows again through the lesion P towards the portion of the artery that is downstream from the lesion, with the duration of the second infusion preferably lying in the range 5 minutes to 10 minutes.

The purpose of the first infusion and of the second infusion is to cause the temperature of the myocardium to tend towards a target temperature (preferably 32° C.) before and after blood begins to flow again via the treated coronary artery.

The values given above for flow rates, durations, temperatures, and pressures may be varied. The control unit is programmed to control the pump and/or the temperature variation unit in order to achieve predetermined setpoints for liquid flow rate, liquid flow durations, and temperature of the liquid flow as a function of the temperature and/or pressure values measured by said probe(s).

1. A device for treating a coronary lesion, the device comprising a coronary angioplasty catheter presenting a distal portion (3a) adapted to penetrate into a coronary artery, the coronary angioplasty catheter presenting a first lumen open at proximal and distal ends of the first lumen to enable a guide wire to pass through the coronary angioplasty catheter and guide it in a coronary artery, the coronary angioplasty catheter also including at least one balloon situated at said distal portion of the catheter, the balloon being connected to a second lumen for controlling the balloon so that the balloon adopts an inflated configuration or a deflated configuration selectively as a function of fluid pressure inside said second lumen, the device being characterized in that it further comprises a heat exchanger, a pump, and at least one first coupling for coupling the device with a liquid reservoir (R) external to the device, the pump being in fluid flow connection with the first coupling, with the heat exchanger, and with the first lumen so as to force a flow of the liquid from the first coupling to the distal end of the first lumen, after passing through the heat exchanger (R).

2. A device according to claim 1, wherein the distal portion of the catheter has a length of at least 1 cm, and the catheter together with its balloon in the deflated configuration has a maximum outside diameter as measured in its distal portion that is less than or equal to 4 mm.

3. A device according to claim 1, wherein the pump presents a pump flow rate and a delivery pressure of at least 1000 kPa for delivering the liquid to the first lumen of the catheter, which flow rate and pressure are adapted to induce a liquid flow rate of at least 10 mL/min via the first lumen.

4. A device according to claim 1, wherein the pump presents a pump flow rate and a delivery pressure of at least 2000 kPa for delivering the liquid to the first lumen of the catheter, which flow rate and pressure are adapted to induce a liquid flow rate of at least 20 mL/min via the first lumen.

5. A device according to claim 1, also including at least one temperature variation element associated with said at least one heat exchanger in order to vary the temperature of the liquid passing through the heat exchanger.

6. A device according to claim 5, wherein, for a liquid flowing through the heat exchanger with a liquid flow rate in the heat exchanger lying in the range 10 mL/min to 40 mL/min, said at least one temperature variation element is adapted to cause the temperature of that liquid to vary with a gradient of up to 40° C.

7. A device according to claim 5, including a control unit and at least one temperature probe arranged to communicate a measured temperature value to the control unit, the temperature value being representative of the temperature of the flowing liquid, the control unit being functionally connected to the pump and to the temperature variation element associated with the heat exchanger to control variation of at least one parameter of the flowing liquid so as to reach a predetermined heat transfer setpoint for transferring heat to the catheter via the flowing liquid, the parameter being selected from the temperature of the flowing liquid and its fluid flow rate.

8. A device according to claim 7, wherein the control unit is arranged to control said at least one temperature variation element associated with said heat exchanger so that the liquid flowing through the first lumen of the catheter presents a temperature over a first period (P1) of at least two minutes that is equal to a first predetermined temperature setpoint value (V1) to within plus or minus 2° C., and a temperature over a second period (P2) of at least two minutes after the first period (P1) that is equal to a second predetermined temperature setpoint value (V2) to within plus or minus 2° C., the second temperature setpoint value (V2) being lower than the first temperature setpoint (V1) by at least 10° C.

9. A device according to claim 8, wherein the first temperature setpoint value (V1) lies in the range 15° C. to 25° C. and the second temperature setpoint value (V2) lies in the range 2° C. to 10° C.

10. A device according to claim 8, wherein the control unit and the temperature variation element are arranged so that the liquid flowing through the heat exchanger can reach the second temperature setpoint value less than one minute after reaching the first temperature setpoint (V1).

11. A device according to claim 7, wherein said temperature value measured by said at least one temperature probe is measured between the heat exchanger and the distal end of the first lumen.

12. A device according to claim 7, wherein the temperature probe is arranged to measure a temperature value upstream from the catheter.

13. A device according to claim 7 wherein the temperature probe is located at the end of a wire arranged to run along and outside the catheter so as to enable the temperature probe to be positioned downstream from the distal end of the catheter.

14. A device according to claim 7, wherein the temperature probe is located at the distal end of the catheter.

15. A device according to claim 5, wherein said at least one temperature variation element includes at least one electrical heater resistance.

16. A device according to claim 5, wherein said at least one temperature variation element includes at least one refrigeration unit comprising a compressor, a condenser, an expander, and an evaporator that are connected together to define a closed circuit in which a refrigerant fluid flows, said heat exchanger being arranged to be thermally coupled with said evaporator in order to cool the liquid flowing through said heat exchanger.
